(19) United States
(12) Patent Application Publication
(10) Pub. No.: US 2013/0210776 A1
(43) Pub. Date: Aug. 15, 2013

(54) COMPOSITION FOR THE TREATMENT OF SUPERFICIAL LESIONS

(71) Applicants: Maikel Hendriks, Amsterdam (NL); Pieternella Bouter, Amsterdam (NL); Katarzyna Kolodziej, Amsterdam (NL)

(72) Inventors: Maikel Hendriks, Amsterdam (NL); Pieternella Bouter, Amsterdam (NL); Katarzyna Kolodziej, Amsterdam (NL)

(73) Assignee: Progressare Medinvest B.V., Amsterdam (NL)

(21) Appl. No.: 13/810,085

(22) PCT Filed: Jul. 15, 2011
(86) PCT No.: PCT/EP2011/062166

§ 371 Date: Apr. 25, 2013

(30) Foreign Application Priority Data
Jul. 15, 2010 (EP) 10169695.3

(51) Int. Cl. A61K 31/19; A61K 31/60

(52) U.S. Cl. CPC A61K 31/19; A61K 31/60
USPC 514/163; 514/557

(57) ABSTRACT

The invention relates to a composition for the treatment of superficial lesions, in particular skin lesions, mucous membrane lesions and/or nail lesions, an applicator comprising such a composition and the use of such a composition. The composition comprises an effective amount of trichloroacetic acid, at least one thickener, and at a physiologically acceptable solvent. And is effective against a plethora of superficial lesions selected from the group consisting of viral warts, verrucae, water warts (molluscum contagiosum), corns and calluses, and skin hyperpigmentation: age spots, solar lentigo, senial lentigo, acne, keratosis pilaris, actinic keratosis, mouth ulcers (canker sores), cold sores, ingrown toenails, onychomycosis, eyelid xanthelasma.

### FIELD OF THE INVENTION

The invention relates to a composition for the treatment of superficial lesions, in particular skin lesions and/or nail lesions and/or lesions in mucous membranes; an applicator comprising such a composition and the use of such a composition.

### BACKGROUND OF THE INVENTION

Various compositions are known for the treatment of skin lesions such as warts, corn and calluses, actinic keratosis, keratosis pilaris, acne, skin hyperpigmentation and/or nail lesions such as ingrown toenails and/or lesions in mucous membranes, such as cold sores and mouth ulcers. Many known compositions are ineffective. Thus, there is a need for more satisfactory treatments.

### OBJECT AND SUMMARY OF THE INVENTION

It is an object of the invention to provide an effective and improved composition for the treatment of skin lesions and/or nail lesions and/or lesions in mucous membranes.

The invention provides a composition for the treatment of skin lesions and/or nail lesions, comprising an effective amount of trichloroacetic acid, at least one thickener, and at least one physiologically acceptable solvent, and optionally other active ingredients. Trichloroacetic acid (TCA) in the composition as described herein, proved to be effective against a plethora of skin lesions, in particular warts, corn and calluses, molluscum contagiosum, acne, skin hyperpigmentation, actinic keratosis as well as nail lesions including ingrown toenails and onchyomycosis, and lesions in mucous membranes such as mouth ulcers and cold sores. Application of trichloroacetic acid was particularly effective against genital warts, and especially against ano-genital warts where it has been studied in comparative trials where its effectiveness was compared with other anti-wart therapies. In addition, TCA application was effective various other skin disorders, in particular: epidermodysplasia veruciformis-skin disorder also caused by HPV virus, acne, to remove acne scars and wrinkles, tattoo removal, eyelid xanthelasma, age spots, senial lentigo and solar lentigo, melasma.

Salicylic acid has a positive effect in treating skin and nails. In addition to that, salicylic acid gives a mild anaesthetic effect. Preferably, salicylic acid is used in concentrations ranging from 1%-20% w/w.

Salicylic acid in combination with TCA showed a synergistic effect against skin lesions and nail lesions. Another advantage is that using a combination of salicylic acid and TCA allows for a composition with a relatively a low concentration of each of salicylic acid and TCA with a similar effect to compositions using only TCA or only salicylic acid. Using the relatively low concentrations of TCA and salicylic acid decreases the chance of skin irritation due to either of these compounds. Moreover, salicylic acid diminished the discomforting burning feeling on skin and nails sometimes experienced by persons treated with products containing substantial amounts of TCA.

The composition as described herein may comprise other ingredients commonly used in cosmetics and pharmaceutical products, such as surfactants, emulsifiers, colorants and perfumes.

An effective amount of TCA is preferably at least 0.1% by weight, more preferably at least 1% w/w. For lower concentrations, the composition is too low to work efficiently. Preferably, the composition comprises TCA in the range of 0.1-50% w/w. Concentrations higher than 50% TCA w/w are also effective, but have an increased risk for complications and should therefore only be applied by skilled persons.

The composition is preferably a fluid, in order to enable easy application and dividing the active ingredient on a skin or nail surface. Fluids include liquid and gel compositions, as well as semi-solid compositions. The composition can be used in for instance a skin peeling treatment, for the medical or cosmetic treatment of skin lesion selected from the group consisting of warts, corn and calluses, and for nail treatments including ingrown toenails. A postulated mechanism of action is that the composition comprising TCA softens the skin or nail, and enables to peel the skin or nail lesion away. For severe lesions, multiple treatments may be needed. Both cosmetic and medical treatments may be performed with the compositions according to the invention. Other active ingredients contributing to the treatment of the lesion may be added. For instance salicylic acid is another component effective in corroding the skin or nails, and may be used as an additional active ingredient in combination with trichloroacetic acid.

The liquid carrier may be a single solvent or mixture of solvents and additives capable of dissolving of mixing with the concentration of TCA used. A preferred liquid carrier is water or a water-based mixture. Water may be mixed with an organic solvent. It is also possible to use a water-free carrier, preferably using easily evaporable solvents. The use of evaporable solvents makes it possible to achieve a relatively high concentration of TCA at a treated location on the skin or nail. Suitable evaporable solvents include methanol, ethanol, propanol, methyl ethyl ketone, acetone, ethyl acetate, and mixtures thereof.

The thickener provides the composition with an increased viscosity, making it easier to focus the active ingredient on the intended location on the skin or nails, preventing to some extend the spreading of the composition to locations where its action is not desired. Also, the thickener improves the time the active ingredient remains on a treated surface, improving the efficacy of the composition. The thickener may be a single compound, but may also comprise a mixture of compounds. The thickener is preferably a gel-forming agent compatible with the liquid carrier used.

Preferably, the composition has a viscosity of at least 3000 mPas as measured using rotary viscometry at 25° C. Viscosity is measured according to the rotating viscosimeter protocol in the European Pharmacopeia Ph. Eur (01/2005:20210) Ph. Eur. 5th edition vol 1, p. 29, chapter 2.2.10. Such compositions have a significant adhering effect on skin and/or nail, allowing for spot treatment. Compositions with viscosities up to about 60.000 mPas are considered to be useful; liquid compositions with higher viscosities are considered to be difficult to handle.

In a preferred embodiment, the composition is a sprayable liquid having a viscosity in the range of about 1.0 mPas to about 2000 mPas at 25° C. Spraying is an easy and fast way to apply the composition to a surface to be treated.

In another preferred embodiment, the composition has a viscosity of at least 5000 mPas as measured at 25° C. according to the rotating viscosimeter protocol in the European Pharmacopeia Ph. Eur (01/2005:20210) Ph. Eur. 5th edition vol 1, p. 29, chapter 2.2.10. Rotating Viscometer Method. Such a viscosity gives the composition a particularly useful adhering effect on skin and nails. Preferably, the composition is a gel composition having a viscosity in the range of about 3000 mPas to 60000 mPas at 25° C. The gel composition is relatively easy to apply, either manually or by using a suitable applicator, and combines a relatively large adhering effect. The gel is particularly suitable to apply the composition selectively to a specific spot, such as a wart.

Preferably, the composition has a viscosity in the range of 450 mPas to 10000 mPas at 25° C., more preferably from 5000 mPas to 10000 mPas at 25° C. Such a composition shows a sufficient adhering effect, while still being relatively easy to apply and process. It is advantageous if the liquid carrier is an aqueous carrier. A water-based composition is relatively easy to prepare, and dissolves TCA well. The water may be mixed with other solvents, for instance C1-C6 alcohols or ketones, and additives such as surfactants. Preferably, the liquid carrier consists for at least 50% of water.

Preferably, the composition has a pH below 4. Low pH compositions appear to provide a better treatment results. In addition, the low pH compositions appear to have an additional effect in suppressing microbiological threats on the skin and nail, including bacteria, fungus and yeast, in particular onchyomycosis (nail fungus).

In a preferred embodiment, the composition comprises at least 1% trichloroacetic acid, more preferably at least 20% w/w trichloroacetic acid, more preferably in the range of 20-50% w/w. Compositions having a concentration of at least 20% w/w show good results in treating skin and nail lesions within a relatively short treatment. Although skin irritations occasionally occur after treatment with TCA concentrations below 50%, TCA concentrations over 50% were found to significantly increase the chance of skin irritations.

It is preferred if the thickener comprises at least thickener selected from polysaccharide thickener, cross-linked acrylic acid polymer and polysiloxanes, or mixtures thereof. These thickeners show a good compatibility with trichloroacetic acid. Polysaccharide thickeners showed in addition to their thickening effect to decrease the chance of skin irritations.

In a preferred embodiment, the thickener comprises at least one thickener selected from the group consisting of amylose, amylopectine, carbopol, silicones, Xanthan gum, agar-agar, polydimethylsiloxane (and mixes thereof), dimethicones, carrageen, cellulose, carboxymethyl cellulose or salts thereof, ethyl cellulose, hydroxypropyl cellulose and methyl cellulose, natural rubber, beeswax, lanolin, petrolatum, paraffin, rosin (and mixtures thereof). The viscosity of the formulation having excellent adhering effects may be achieved using these thickeners. Carbopol is a brand name for cross-linked polymers of acrylic acid, and includes the commercially available products Carbopol 71G NF, Carbopol 971P NF, Carbopol 974P NF, Carbopol 934P NF, Carbopol 980P NF, Carbopol 981P NF, Carbopol 5984EP, Carbopol ETD 2020 NF, Carbopol 934 NF, Carbopol 934P NF, Carbopol 940 NF, Carbopol 941 NF, Carbopol 1342 NF, Pemulen TR-1 NF, Pemulen TR2-NF, Noveon AA-USP, and Carbopol Ultrez 10 NF.

Preferably, the thickener remains essentially stable in the presence of trichloroacetic acid. Some thickeners are degraded in the presence of TCA, resulting in a coloured product, which may appear less attractive to some persons. Also, the color of a solution may be difficult to control in case a colorant was used. A colourless composition is generally considered to be visually more attractive and therefore more desirable.

Most preferably, the thickener comprises carbopol. Carbopol shows an excellent stability in the presence of TCA, allowing for colorless compositions, or excellent color control in case the composition comprises a colorant. Carbopol polymers, also called carbomers, are polymers of acrylic acid cross-linked with polyalkenyl ethers or divinyl glycol.

Carbopol allows for an excellent control of the viscosity of the formulation. Preferably, the composition comprises at least 0.5% w/w carbopol, preferably in the range of 0.5-3% w/w. Compositions comprising 0.5%-1% w/w carbopol are excellent for sprayable products, products comprising 1.5-2.5% w/w form a well-applicable gel. Products comprising carbopol more than 3% w/w may become too rigid for easy processing and application.

In a preferred embodiment, the composition comprises a combination of carbopol and glycerin. Although glycerin is by itself not a thickener, the addition of glycerin to a composition according to the invention using carbopol yields an increase in viscosity. Thus the cost price for a formulation with a relatively high viscosity may be reduced by using less of the relatively expensive carbopol by adding the relatively cheap glycerin. Preferably, glycerin is added in an amount ranging from approximately 1% w/w to approximately 20% w/w. As an additional effect, glycerin was found to reduce the chance of skin irritation by TCA.

The invention also provides an applicator device comprising a composition according to any of the preceding claims. The applicator may be design for efficient application of the composition depending on its intended purpose (for instance skin of nail treatment), and considering the viscosity of the formulation. The applicator device may for instance be a pen applicator, a brush applicator, roll-on applicator or a spray applicator, a tube, roller, glass jar with the spatula, hydrocolloid patch, plaster, cryo-spray, lipstick pen, droplets dispenser (with pipette), cryo-spray or a cream dispenser.

The invention further provides the use of a composition according to the invention for the cosmetic treatment of the human skin.

Preferably, the composition is used for a skin peeling treatment or the treatment of a skin lesion selected from the group consisting of warts, corn and calluses. The compositions are particularly effective against warts and related lesions, including viral warts, verrucae, and water warts (molluscum contagiosum). These treatments are typically considered cosmetic treatments rather than medical treatments.

The invention also provides the use of a composition as described herein for the cosmetic treatment of nails. In particular nail deformations (for instance as a result from onchyomycosis) and ingrown toenails may be treated effectively.

The invention further provides the use of a composition as described herein for the treatment of lesions in mucous membranes, in particular mouth ulcers and cold sores.

The invention further provides the use of a composition as described herein for the cosmetic treatment of superficial lesions selected from the group consisting of viral warts, verrucae, water warts (molluscum contagiosum), corns and calluses, and age spots, solar lentigo, senial lentigo, acne, mouth ulcers (canker sores), cold sores ingrown toenails

### DESCRIPTION OF PREFERRED EMBODIMENTS

The invention will now be further elucidated by the following non-limiting examples.

### Methods

TCA is the abbreviation for trichloroacetic acid. Ingredients were mixed using conventional mixing techniques. pH of the compositions was determined at room temperature (25° C.) using a commercially available digital pH meter and/or universal pH indicator test strips.

Viscosity was measured in a commercially available Brookfield RV rotational viscosity meter at room temperature (25° C.), at 20 rpm, according to the method described in Ph. Eur. 5th edition vol 1, p. 29, chapter 2.2.10. Rotating Viscometer Method.

### EXAMPLES

The next tables show various compositions according to the invention, prepared by mixing the appropriate ingredients using regular methods. The compositions described herein may be applied by known or newly developed applicator devices, for instance in the form of a spray, pen, tube, glass jar with the spatula, plaster, cream dispenser.

### Example 1

### Spray Compositions

| Ingredient % | | | |
|---|---|---|---|
| (w/w) | Spray 1 | Spray 2 | Spray 3 |
| TCA | 20 | — | 20 |
| Salicylic acid | — | 2 | 2 |
| carbopol | 0.5 | 0.5 | 0.5 |
| water | Fill to 100% | Fill to 100% | Fill to 100% |
| pH | 2 | 3 | 3 |
| Viscosity (mPas) | 5 | 5 | 5 |

These compositions have a relatively low viscosity and is therefore fast and easy to apply by a spray applicator, either from a canister using a propellant gas, or a manual pump system. The low viscosity makes it easy to treat a relatively large skin or nail area. The carbopol thickener ensures that the liquid composition adheres to the treated area. In this example, the commercially available Carbopol ETD 2020NF was used as a thickener, but other alternative thickeners or mixtures of thickeners are possible.

These compositions are particularly suitable for treating calluses and for the whole body in light and medium-deep chemical peel treatments. In the treatment of corns or calluses, or other thickened skin lesions, the composition is sprayed on the lesions and allowed to work for at least 1 hour, preferably 3-8 hours. Afterwards, remainders of the composition may be washed off using water.

For chemical peels, the composition is applied over a treated skin area (preferably by spraying) and allowed to react for 5-30 minutes, after which the composition may be washed off using water. Optionally, a neutralizing pH buffer or other neutralizing agent may be used in case a burning sensation and skin irritation occurs, in particular for the compositions comprising TCA.

Spray 1 (TCA) showed better and faster results than spray 2 (salicylic acid). Spray 3, using a combination of TCA and salicylic acid, showed a better and faster effect than either spray 1 or spray 2, and users experienced a diminished burning feeling and lower chance of skin irritation compared to the formulations comprising TCA without salicylic acid.

### Example 2

### Hard Gel Compositions

| Ingredient % | | | |
|---|---|---|---|
| (w/w) | Gel 1 | Gel 2 | Gel 3 |
| TCA | 40 | 0 | 40 |
| Salicylic acid | — | 2 | 2 |
| carbopol | 2 | 2 | 2 |
| glycerine | 12 | 12 | 12 |
| water | Fill to 100% | Fill to 100% | Fill to 100% |
| pH | 2 | 3 | 2 |
| viscosity | 8000 | 8000 | 8000 |

The gel compositions are easily applied locally, either manually or by an applicator. The carbopol thickener ensures that the composition has a sufficient viscosity to retain the active ingredient at the selected area to be treated (for instance a toe nail), while diminishing spreading of the active ingredient over to areas that do not need treatment. This lowers the chance of unnecessary skin irritation outside the area or spot that needs treatment. Varying the carboxymethylcellulose amount from 0.5-2% w/w yields compositions with a viscosity ranging from 5000-10000 MPas.

It was discovered that adding glycerine enhances the thickening effect of carbopol and lowers the chance for skin irritation.

These gel compositions are particularly suitable for local treatment of lesions, in particular warts, corns, calluses and ingrown toenails. The compositions are applied to the lesions and allowed to work for at least 10 minutes, preferably a number of hours, and will not necessarily have to be washed off, unless skin irritations occur.

Gel 1 (TCA) showed better and faster results than gel 2. Gel 3, using a combination of TCA and salicylic acid, showed a better and faster effect than either gel 1 or gel 2, and users experienced a diminished burning feeling and lower chance of skin irritation compared to the formulations comprising TCA without salicylic acid.

### Example 3

### Gel Composition Comparison

| Ingredient % (w/w) | Gel 4 | Gel 5 | Gel 6 |
|---|---|---|---|
| TCA | 40 | 0 | 40 |
| Salicylic acid | — | 2 | 2 |
| carboxymethylcellulose | 4 | 4 | 4 |
| water | Fill to 100% | Fill to 100% | Fill to 100% |
| pH | 2 | 3 | 2 |
| viscosity | 8000 | 8000 | 8000 |

The gel composition is easily applied locally, either manually or by an applicator. The carboxymethylcellulose thickener ensures that the composition has a sufficient viscosity to retain the active ingredient at the selected area to be treated (for instance a toe nail), while diminishing spreading of the active ingredient over to areas that do not need treatment. Carboxymethylcellulose and other cellulose derivatives are particularly suitable as thickeners for TCA compositions, as cellulose derivatives were found to lower the chance of skin irritation by TCA. The amount of carboxymethylcellulose or an equivalent cellulose derivative may be varied depending on the desired viscosity. Varying the carboxymethylcellulose amount from 2-5% w/w yields compositions with a viscosity ranging from 5000-10000 MPas.

These compositions are particularly suitable for treating warts, corns and calluses. The composition are applied to the lesions and allowed to work for at least 10 minutes, preferably at least a 1 hour.

Gel 4 (TCA) showed better and faster results than gel 5. Gel 6, using a combination of high concentration of TCA and salicylic acid, showed a better and faster effect than either gel 4 or gel 5, and users experienced a diminished burning feeling and lower chance of skin irritation compared to the formulations comprising TCA without salicylic acid.

Various excellent water-based compositions without salicylic acid could also be obtained, shown in Table 4. This table shows that the viscosity of the fluid gel formulations depends not only on the amount of thickener but also on the amount of active ingredient trichloroacetic acid.

### Example 4

### Water-Based Gel Compositions

| Ingredient % | | | |
|---|---|---|---|
| (w/w) | Gel 7 | Gel 8 | Gel 9 |
| TCA | 40 | 20 | 20 |
| Carbopol polymer | 1.5 | 1.75 | 2 |
| (preferably | | | |
| Carbopol ® ETD | | | |
| 2020NF) | | | |
| water | Fill to 100% | Fill to 100% | Fill to 100% |
| pH | <2 | <2 | <2 |
| viscosity | 6000-8000 | 3000-4000 | 4000-6000 |

Gel 4 (TCA) is very efficient after a single application to a lesion to be treated. After locally applying the gel and letting the composition in place overnight, the composition causes an efficient peeling of the treated skin lesion, The gel can be effectively used in pen applicators for instance large warts removal, wherein the viscosity of the gel provides well sized gel drops allows that can be applied precise on the spot to be treated, thus providing a user-friendly applications. Gels 8 and 9 (TCA) can also be used against several types of skin lesions, and are particularly effective on calluses, corns, small warts or other smaller skin lesions. In addition, gel 8 and gel 9 are also very convenient to be used in pen applicators.

### Example 5

### Water-Free Gel Composition

| Ingredient % (w/w) | Gel 10 | Gel 11 | Gel 12 |
|---|---|---|---|
| TCA | 30 | — | 30 |
| Salicylic acid | — | 2 | 2 |
| hydroxypropylcellulose | 2 | 2 | 2 |
| Castor oil | 4 | 4 | 4 |
| Methanol | 20 | 20 | 20 |
| Acetone | Fill to 100% | Fill to 100% | Fill to 100% |
| pH* | 2 | 3 | 2 |
| viscosity | 3000 | 3000 | 3000 |

*as determined by using water-wetted multipurpose pH test strips.

The gel composition is easily applied locally, either manually or by an applicator. The hydroxypropylcellulose thickener ensures that the composition has a sufficient viscosity to retain the active ingredient at the selected area to be treated (for instance a toe nail), while diminishing spreading of the active ingredient over to areas that do not need treatment. Hydroxypropylcellulose and other cellulose derivatives are particularly suitable as thickeners for TCA compositions. The use of evaporable solvents makes it possible to achieve a relatively high concentration at a treated location on the skin or nail. Suitable evaporable solvents include methanol, ethanol, propanol, methyl ethyl ketone, acetone, ethyl acetate, and mixtures thereof.

These compositions are particularly suitable for treating warts, calluses, corns and ingrown nail. The compositions are applied to the lesions and allowed to work for at least 10 minutes, preferably at least 1 hour.

Gel 10 (TCA) showed better and faster results than gel 11 (salicylic acid). Gel 12, using a combination of TCA and salicylic acid, showed a better and faster effect than either gel 10 or gel 11, and users experienced a diminished burning feeling and lower chance of skin irritation compared to the formulations comprising TCA without salicylic acid.

### Example 6

### Alternative Gel Compositions

| Ingredient % (w/w) | Gel 13 | Gel 14 | Gel 15 |
|---|---|---|---|
| TCA | 3 | 3 | 2 |
| Sodium gluconate | 1 | 1 | 0.4 |
| Sodium benzoate | 0.2 | 0.2 | 0.2 |
| Potassium sorbate | 0.2 | 0.2 | 0.2 |
| Eusolex ® T-oleo | 4.5 | — | |
| Eusolex ® 4360 | 1.5 | — | |
| Eusolex ® 2292 | 1 | — | |
| PEG-40 Hydrogenated | 5 | — | |
| Castor Oil | | | |
| Glycerine | 6 | 3 | 5 |
| Carboxymethylcellulose | 5 | 2.5 | |
| Carbopol polymer | — | — | 2.5 |
| (preferably Carbopol ® | | | |
| ETD 2020NF) | | | |
| water | Fill to 100% | Fill to 100% | Fill to 100% |
| pH | 4 | 3 | 2 |
| viscosity | 6000-7000 | 6000-8000 | 7000-10 000 |

Gel 13 is particularly suitable for treatment of small (more superficial) skin lesions with additional UVA/UVB filter protection. Gel 14 and Gel 15 are hard gels, which can be used for small (superficial) skin lesions and can be used in a form of pen applicator.

### Example 7

| Ingredient % (w/w) | Gel 16 | Gel 17 |
|---|---|---|
| TCA | 0.4 | 1 |
| Olive leaf Lactobacillus extract | 5 | 5 |
| Glycerine | 5.6 | 4 |
| Allantoin | 0.1 | 0.1 |
| Urea | 1.0 | 1.0 |
| Dimethyl isosorbide | 1.5 | 1.5 |
| Sodium hydroxide | 2.6 | 3.0 |
| Carbopol polymer (preferably | 0.9 | 1.5 |
| Carbopol ® ETD 2020NF) | | |
| D-Panthenol | 1 | 1 |
| Potassium sorbate | 0.2 | 0.2 |
| Xanthan gum | 0.4 | 0.15 |
| Sodium benzoate | 0.02 | 0.02 |
| water | Fill to 100% | Fill to 100% |
| pH | 4.5-5.5 | 4.5-5.5 |
| viscosity | 4000-8000 | 6000-15000 |

Gel 16 and 17 are preferably to be used on actinic keratosis and acne to peel off the external layer of sebum and keratinized, dead epithelium. Gels 16 and 17 are particularly suitable for use on delicate skin, for example on facial areas.

### Example 8

| Ingredient % (w/w) | Gel 18 |
|---|---|
| TCA | 1-50 |
| Natural rubber | 10-28.5 |
| Lanolin | 2 |
| Petrolatum | 7.5 |
| Butylated hydroxytoluene | 0.2 |
| Zinc oxide | 0-28.3 |
| Rosin | 15-30.5 |
| Abietic Resin | 3 |

Gel 18 is particularly suitable as a piercing composition of wart and/or corn and/or comedone and/or cold sore, using a plaster as the applicator device. The piercing composition has direct contact with the skin of wart or callus. The piercing composition may be applied as a spot on the band aid and can be used alone or in combination with the encircling padding.

### Efficacy Study

A study was performed to demonstrate effectiveness of TCA (trichloro-acetic acid) in treatment of Warts, in particular verruca vulgaris and verruca plantaris. For the studies, a composition using 40% w/w trichloroacetic acid as described herein (Gel 7) applied using a wart pen applicator.

### Methods

Fifty one participants (aged 11-53) with warts on hands and feet were recruited to the study. Participants were instructed to apply TCA gel twice daily to the lesions for 4 consecutive days followed by a 4-day resting period. Follow-up doctor visits were scheduled every week, for maximum 5 treatment weeks.

### Results

In total, 79 warts were treated and 65% of warts were successfully cleared off within the trial time (51 out of 79; 25 foot warts and 26 hand warts) Out of the 79 treated warts, 40 occurred on the hands and 39 occurred on the feet. The success rates for hand and foot wart clearance were similar: 62% and 66% respectively. In the 51 participants, more than 50% of warts were completely removed upon completion of the study. In total, 76% of patients had either partially or totally succeeded in wart(s) removal. It is anticipated that a prolonged treatment period after the 5-week trial, could also have removed the more persistent warts.

### CONCLUSIONS

Out of 79 total treated warts 65.5% dropped off, showing a good efficiency. On average the treatment requires minimum 3 four day treatment periods. Majority of warts that cleared within less than 3 four day treatment periods were hand warts. As noted by patients and dermatologists, foot warts are more difficult to treat and take longer to drop off. No unexpected side effects were noted.

1. Composition for the treatment of superficial lesions, in particular skin lesions, mucous membrane lesions and/or nail lesions, comprising
trichloroacetic acid,
at least one thickener (for increasing the viscosity of the solvent), and
at least one physiologically acceptable liquid carrier, and optionally other active ingredients.

2. Composition according to claim 1, wherein the composition has a viscosity of at least 3 mPas as measured using rotary viscometry at 25° C.

3. Composition according to claim 2, wherein the composition has a viscosity of at least 3000 mPas at 25° C.

4. Composition according claim 2, wherein the composition has a viscosity in the range of 5000 mPas to 10000 mPas at 25° C.,

5. Composition according to claim 1, wherein the liquid carrier is an aqueous carrier.

6. Composition according to claim 1, wherein the composition has a pH below 4.

7. Composition according to claim 1, wherein the composition comprises at least 1% w/w trichloroacetic acid, preferably at least 20% w/w trichloroacetic acid, more preferably in the range of 20-50% w/w.

8. Composition according to claim 1, wherein the composition comprises a combination of trichloroacetic acid and salicylic acid.

9. Composition according to claim 1, wherein the thickener comprises at least one thickener selected from polysaccharide thickener, cross-linked acrylic acid polymer and polysiloxanes, or mixtures thereof, natural rubber, glycerin, petrolatum, paraffin, lanolin, beeswax, rosin, Xanthan gum, amylose, amylopectine, carbopol, cellulose, Carboxymethyl cellulose or salts thereof, ethyl cellulose, glycerin, hydroxypropyl cellulose and methyl cellulose, or mixtures thereof.

10. Composition according to claim 1, wherein the thickener comprises cross-linked polymers of acrylic acid (carbopol).

11. Composition according to claim 10, wherein the composition comprises at least 0.5% w/w cross-linked polymers of acrylic acid, preferably in the range of 0.5-3% w/w.

12. Composition according to claim 10, wherein the composition comprises a combination of cross-linked polymers of acrylic acid and glycerin.

13. Applicator device comprising a composition according to claim 1.

14. A method to treat human skin, nails and/or mucous membranes comprising administering to the human a composition according to claim 1 to treat the human skin, nails and/or mucous membranes.

15. The method according to claim 14 for the cosmetic treatment of superficial lesions selected from the group consisting of viral warts, verrucae, water warts (molluscum contagiosum), corns and calluses, and skin hyperpigmentation: age spots, solar lentigo, senial lentigo, acne, keratosis pilaris, actinic keratosis, mouth ulcers (canker sores), cold sores, ingrown toenails, onychomycosis, and eyelid xanthelasma.
